(19) United States
(12) Patent Application Publication
(10) Pub. No.: US 2015/0237452 A1
(43) Pub. Date: Aug. 20, 2015

(54) SYSTEMS AND METHODS FOR FITTING AN AUDITORY PROSTHESIS

(71) Applicant: Filiep Vanpoucke, Macquarie University (AU)

(72) Inventor: Filiep Vanpoucke, Macquarie University (AU)

(21) Appl. No.: 14/542,301

(22) Filed: Nov. 14, 2014

Related U.S. Application Data
(60) Provisional application No. 61/940,072, filed on Feb. 14, 2014.

(51) Int. Cl. H04R 25/00; A61N 1/36

(52) U.S. Cl. CPC H04R 25/70; A61N 1/36032; H04R 25/30

(57) ABSTRACT

System and methods for fitting an auditory prosthesis include testing a subset of sound channels for the audio prosthesis. Adjustment values are determined for the tested sound channels, which can be electrodes in a cochlear implant. The adjustment values are efficiently determined by using a mapping function. The determined adjustment values on selected channels are then input into an interpolation function or algorithm to determine adjustment values for non-tested sound channels. The use of interpolation provides for a more efficient fitting process.

### CROSS-REFERENCE TO RELATED APPLICATIONS

This application claims priority to and the benefit of U.S. Provisional Patent Application No. 61/940,072, filed Feb. 14, 2014, entitled “SYSTEMS AND METHODS FOR FITTING AN AUDITORY PROSTHESIS,” the disclosure of which is incorporated by reference herein in its entirety.

### BACKGROUND

Audio prostheses are generally fitted to individual recipients to ensure proper functioning of the prosthesis and to improve audio detection for the recipient. However, due to the differences between audio prostheses and the different conditions of each recipient, the fitting process is often a difficult task. It is with respect to this general environment that embodiments of the present disclosure have been contemplated.

### SUMMARY

Embodiments of the present disclosure relate to systems and methods for performing a fine tuning fitting of an auditory prosthesis by testing a subset of sound channels for the audio prosthesis. In embodiments, adjustment values are determined for the tested sound channels. The determined adjustment values are then input into an interpolation function or algorithm to determine adjustment values for non-tested sound channels.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

The same number represents the same element or same type of element in all drawings.

FIG. 1 is an example of a cochlear implant system 100.

FIG. 2 is a simplified side view of an internal component 244 having a stimulator/receiver unit 202 which receives encoded signals from an external component of the cochlear implant system.

FIG. 3 is an embodiment of a Pure-Tone Algorithm (PTA) profile 300 that can be generated during a testing procedure of an auditory prosthesis.

FIG. 4 is an embodiment of a method 400 of performing a fine tune fitting of an auditory prosthesis using interpolation.

FIG. 5 is an exemplary embodiment of a graph 500 illustrating the recalculation a T-level for an electrode in a cochlear implant.

FIG. 6 is yet another exemplary embodiment of a method 600 for adjusting an auditory prosthesis using interpolation.

FIG. 7 illustrates one example of a suitable operating environment 700 in which one or more of the present examples can be implemented.

FIG. 8 is an embodiment of a network 800 in which the various systems and methods disclosed herein may operate.

### DETAILED DESCRIPTION

Cochlear implants generally include a stimulating assembly implanted in the cochlea to deliver electrical stimulation signals to the auditory nerve cells, thereby bypassing absent or defective hair cells. The electrodes of the stimulating assembly differentially activate auditory neurons that normally encode differential pitches of sound.

Cochlear implants have traditionally comprised an external speech processor unit worn on the body of the recipient and a receiver/stimulator unit implanted in the mastoid bone of the recipient. The external speech processor detects external sound and converts the detected sound into a coded signal through a suitable speech processing strategy. The coded signal is sent to the implanted receiver/stimulator unit via a transcutaneous link. The receiver/stimulator unit processes the coded signal to generate a series of stimulation sequences which are then applied directly to the auditory nerve via a series-arrangement or an array of electrodes positioned within the cochlea.

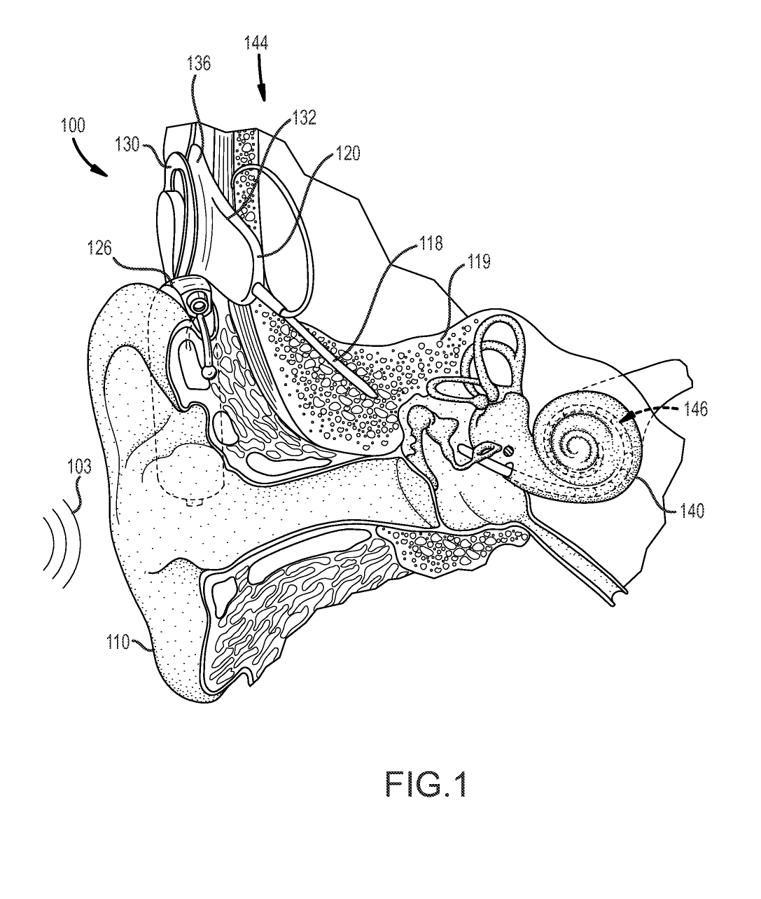

Referring to FIG. 1, cochlear implant system 100 includes an implantable component 144 typically having an internal receiver/transceiver unit 132, a stimulator unit 120, and an elongate lead 118. The internal receiver/transceiver unit 132 permits the cochlear implant system 100 to receive and/or transmit signals to an external device 126 and includes an internal coil 136, and preferably, a magnet (not shown) fixed relative to the internal coil 136. These signals generally correspond to external sound 103. Internal receiver unit 132 and stimulator unit 120 are hermetically sealed within a biocompatible housing, sometimes collectively referred to as a stimulator/receiver unit. The magnets facilitate the operational alignment of the external and internal coils, enabling internal coil 136 to receive power and stimulation data from external coil 130. Elongate lead 118 has a proximal end connected to stimulator unit 120, and a distal end implanted in cochlea 140. Elongate lead 118 extends from stimulator unit 120 to cochlea 140 through mastoid bone 119.

In certain examples, external coil 130 transmits electrical signals (e.g., power and stimulation data) to internal coil 136 via a radio frequency (RF) link, as noted above. Internal coil 136 is typically a wire antenna coil comprised of multiple turns of electrically insulated single-strand or multi-strand platinum or gold wire. The electrical insulation of internal coil 136 is provided by a flexible silicone molding. Various types of energy transfer, such as infrared (IR), electromagnetic, capacitive and inductive transfer, can be used to transfer the power and/or data from external device to cochlear implant.

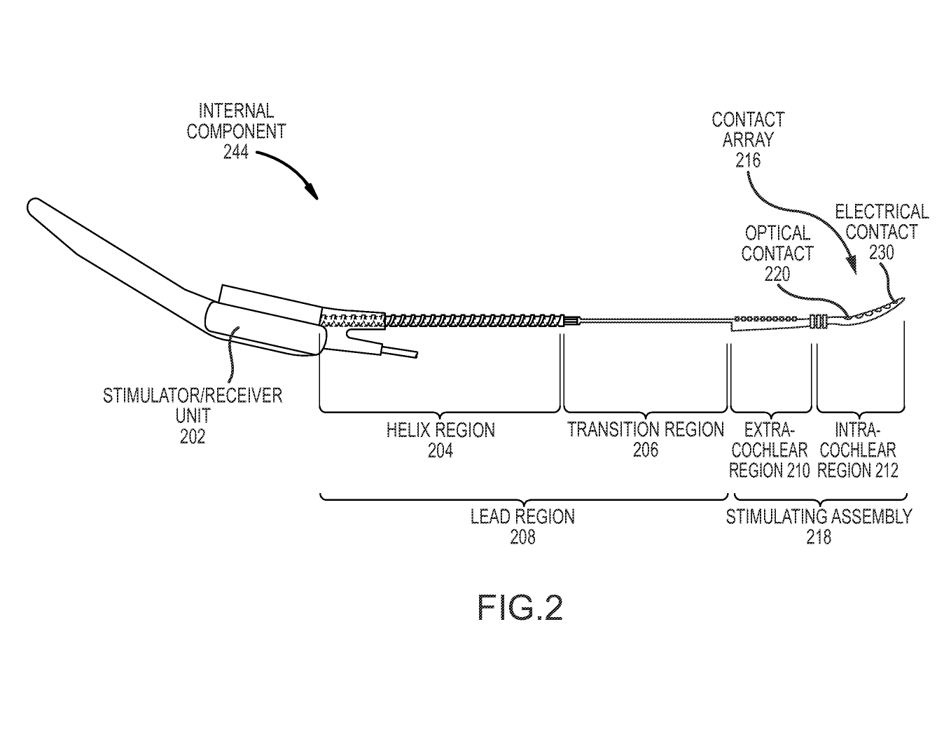

FIG. 2 is a simplified side view of an internal component 244 having a stimulator/receiver unit 202 which receives encoded signals from an external component of the cochlear implant system. Internal component 244 terminates in a stimulating assembly 218 that comprises an extra-cochlear region 210 and an intracochlear region 212. Intra-cochlear region 212 is configured to be implanted in the recipient's cochlea and has disposed thereon a contact array 216. In the present example, contact array 216 comprises both optical contacts 220 and electrical contacts 230. In embodiments, the electrical contacts 230 can be one or more electrodes. Other embodiments can employ optical stimulation alone or in conjunction with electrical or other stimulation mechanisms.

There are a variety of types of intra-cochlear stimulating assemblies including short, straight and perimodiolar. Perimodiolar stimulating assembly 218 is configured to adopt a curved configuration during and or after implantation into the recipient's cochlea. To achieve this, in certain arrangements, stimulating assembly 218 is pre-curved to the same general curvature of a cochlea. Such examples of stimulating assembly 218, are typically held straight by, for example, a stiffening stylet (not shown) or sheath which is removed during implantation, or alternatively varying material combinations or the use of shape memory materials, so that the stimulating assembly can adopt its curved configuration when in the cochlea. Other methods of implantation, as well as other stimulating assemblies which adopt a curved configuration, can be used.

Stimulating assembly 218 can also be a non-perimodiolar stimulating assembly. For example, stimulating assembly 218 can comprise a straight stimulating assembly or a mid-scala assembly which assumes a midscale position during or following implantation.

Alternatively, stimulating the stimulated assembly can be a short electrode implanted into at least in basal region. The stimulating assembly can extend towards apical end of cochlea, referred to as cochlea apex. In certain circumstances, the stimulating assembly can be inserted into cochlea via a cochleostomy. In other circumstances, a cochleostomy can be formed through round window, oval window, the promontory or through an apical turn of cochlea.

Internal component 244 further comprises a lead region 208 coupling stimulator/receiver unit 202 to stimulating assembly 218. Lead region 208 comprises a region 204 which is commonly referred to as a helix region, however, the required property is that the lead accommodate movement and is flexible, it does not need to be formed from wire wound helically. Lead region also comprises a transition region 206 which connects helix region 204 to stimulating assembly 218. As described below, optical and/or electrical stimulation signals generated by stimulator/receiver unit 202 are delivered to contact array 216 via lead region 208. Helix region 204 prevents lead region 208 and its connection to stimulator/receiver 202 and stimulating assembly 218 from being damaged due to movement of internal component 144 (or part of 144) which can occur, for example, during mastication.

More recently, the external speech processor and implanted stimulator unit can be combined to produce a totally implantable cochlear implant (TICI) capable of operating, at least for a period of time, without the need for an external device. An alternative to the TICI is the mostly implantable cochlear implant (MICI) in which a battery is implanted and all or some of the sound processing is moved to the implant and a smaller (or very small) external processor contains the microphone and the capability to wirelessly transmit information to the implant via RF signals.

After surgical implantation, a cochlear implant system needs to be fitted for each recipient individually. The fitting process involves the creation of a MAP which defines the specific characteristics of the electrical signal used to stimulate the electrodes of implanted electrode array. Normally, two values need to be set for each stimulating electrode of the array of electrodes. These values are referred to as the threshold level (also referred to as the “THR” or “T level”), and the maximum comfort level (also referred to as the “Most Comfortable Loudness level,” “MCL,” “M level,” “C level,” “Maximum Comfortable Loudness,” or simply “comfort level”). Threshold levels reflect the amount of electrical stimulation at which a hearing percept becomes perceptible. Comfort levels indicate the amount of electrical stimulation at which the hearing percept is loud but comfortable to the recipient.

As the recipient adjusts to the cochlear implant, the perceived performance can change as the recipient's brain adapts to hearing new sounds. As the recipient's brain adapts, the settings determined during the initial fitting may no longer be the most desirable settings for the recipient. However, after the initial programming, the options for making adjustments are limited. Frequently, it is not practical or economic for the recipient to re-visit the clinic and repeat the original programming process. This is because the process is time consuming and difficult.

Sometimes, the recipient is given the ability to adjust the overall channel gain or volume so as to increase or decrease perceived loudness. Increased loudness, however, may distort sound perception. Thus, while the sounds heard by the recipient may be louder, for critical sounds such as spoken language, the recipient's understanding may actually decrease. This is undesirable, as hearing and understanding spoken language is often the most desired outcome involved in the use of an auditory prosthesis. Thus, increase in perceived volume is often at odds with language understanding.

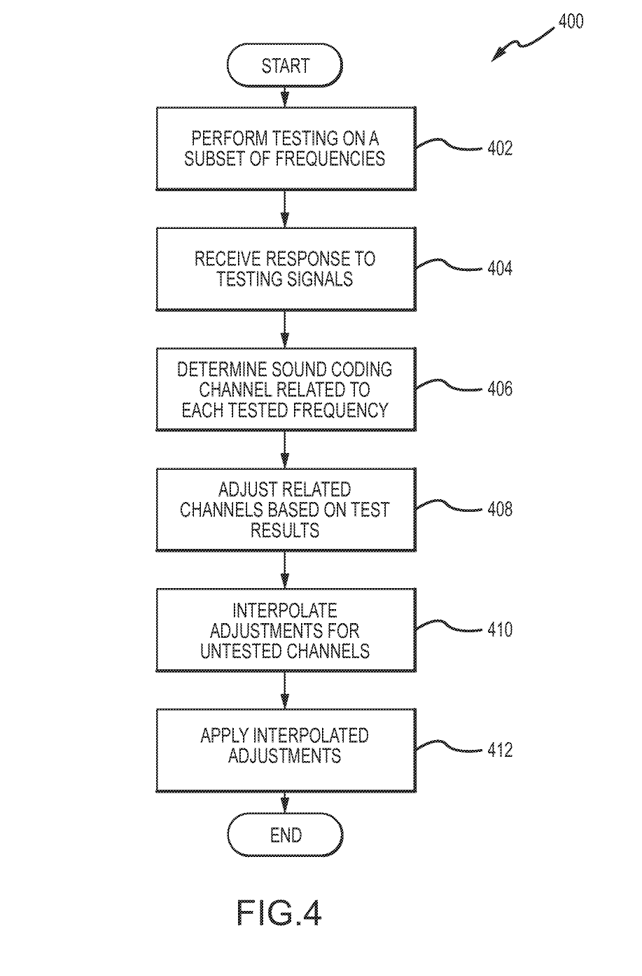

FIG. 4 is an embodiment of a method 400 of adjusting an earlier fitted cochlear implant. For example, the method 400 can be performed on a recipient after an initial usage period that gives the recipient an opportunity to adjust to the device.

Flow begins at operation 402 where a subset of pure tone audio frequencies is tested. For each one of the subset of pure tone audio test frequencies, a pure tone acoustic signal is presented to the recipient. The intensity of the acoustic stimulation is gradually increased (in dB) until the recipient hears a percept.

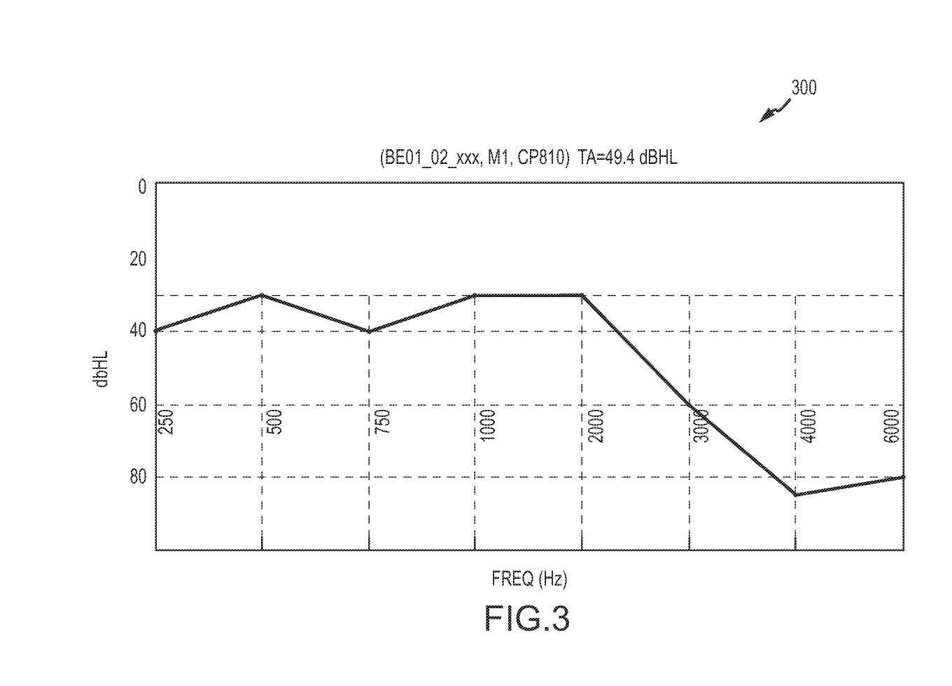

Flow continues to operation 404, where one or more responses indicative of a testing signal being detected are received. The testing results, which include a threshold intensity level for each frequency channel, is recorded to a graph as shown for example in the Pure-Tone Audiogram (PTA) profile (300) of FIG. 3. In the example of FIG. 3, the recipient exhibited target detection levels (e.g., detection around 30 dBHL) for frequencies of 250 Hz, 500 Hz, 750 Hz, 1,000 Hz, and 2,000 Hz. The recipient's detection levels significantly drop at higher frequencies. As illustrated in PTA profile 300, the detection level for sounds around 4,000 Hz and 6,000 Hz is around 80 dBHL.

Flow continues to operation 406, where one or more sound coding channels are determined for each one of the subset of pure tone audio frequencies. For example, a sound channel can be one or more electrodes related to each of the tested frequencies. Preferably, each one of the subset of pure tone audio frequencies relates to a sound coding channel in the device. The configuration of sound coding channels in the device can depend on the number of active channels and the frequency allocation table (FAT).

Upon determining the related sound coding channels, flow continues to operation 408 where, depending on the test results generated at operation 404, the one or more tested sound channels are adjusted to fine tune the prosthesis. In embodiments, a determination is made to adjust the sound channel based on the test results. In an exemplary embodiment, an optimal detection level is 30 dBHL or better. In such embodiments, sound channels that do not meet the ideal recipient detection level are adjusted at operation 408. For example, referring to the exemplary PTA profile 300, sound channels related to the 3,000 Hz, 4,000 Hz, and 6,000 Hz frequencies are adjusted at operation 408 to improve the detection level from near 80 dBHL to a detection level closer to 30 dBHL (e.g., a threshold). In embodiments where the tested auditory prosthesis is a cochlear implant, the sound channel is an electrode related to the tested frequency. For example, referring again to PTA profile 300, a first electrode corresponding to 4,000 Hz and a second electrode corresponding to 6,000 Hz are determined at operation 404 and then adjusted at operation 406 to improve detection levels for the particular frequencies. The adjustment at operation 404 can be made by way of recalculating the T-level for the corresponding sound channel.

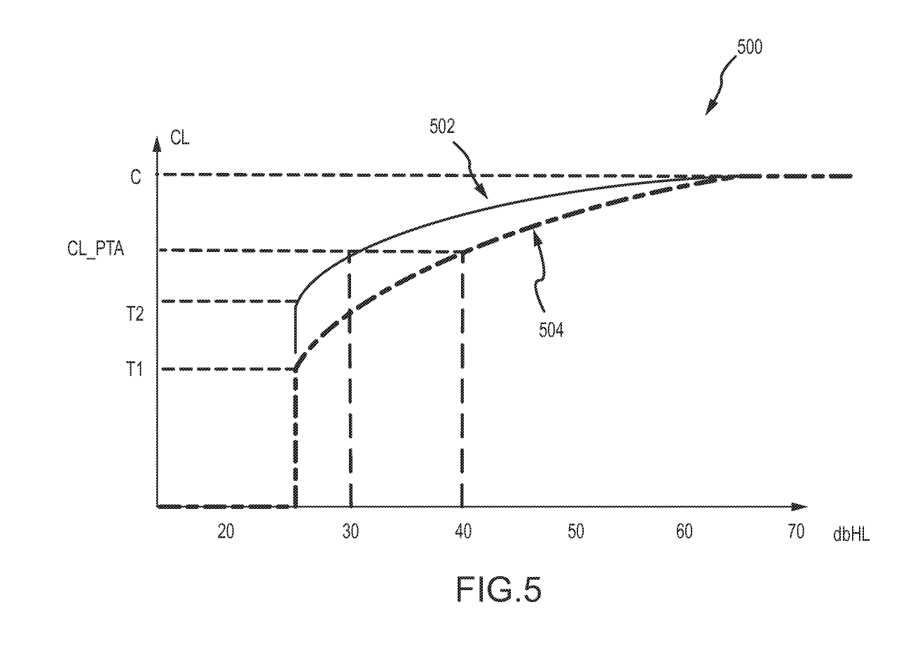

FIG. 5 is an exemplary graph 500 illustrating the recalculation of a T-level for an electrode in a cochlear implant. In the embodiment illustrated by the graph 500, a hearing test was performed on a recipient. In the exemplary test, the recipient detected or otherwise responded to the sound (denoted by line 502) at 40 dBHL, e.g., the amount of acoustic energy required for the recipient to detect the sound played during the test.

As previously described, an optimal detection level is at 30 dBHL or better. In the described embodiment, the electrical stimulation level that has been detected by the recipient at 40 dBHL is denoted CL_PTA. In the exemplary embodiment, this electrical stimulation level is the minimal current required for the brain of the recipient to detect a signal.

To obtain an aided threshold at 30 dBHL, the mapping parameters for the particular electrode can be adjusted to stimulate at level CL_PTA for an acoustic input of 30 dBHL. In other words, an acoustic-to-electrical mapping procedure is performed that maps to 30 dBHL the electrical stimulation that generated was originally generated at 40 dBHL. This causes the same amount of electrical stimulation to be delivered at 30 dBHL, thus improving the recipient's detection level.

Referring back to FIG. 4, upon adjusting the sound channels based on the testing results, flow continues to operation 410 where one or more adjustments are interpolated for sound channels that were not tested. As such, non-tested sound channels (e.g., electrodes in a cochlear implant) are adjusted based upon one or more adjusted values of the tested sound channels. Because only a subset of the frequencies capable of being processed by the auditory prosthesis was tested, adjustments for the coded channels not tested can be interpolated to increase the detection level of the non-tested channels of the auditory prosthesis. In embodiments, mathematical interpolation is employed. The known adjustments made for the tested sound channels are provided as input to a mathematical interpolation function to determine adjustments for the non-tested channels. In one embodiment, a linear interpolation is used at operation 410. In other embodiments, depending on the number of inputs used to make adjustments for the tested channels, other types of mathematical interpolation can be employed, such as bilinear interpolation, trilinear interpolation or the like.

Upon interpolating adjustments for the non-tested sound channels, flow continues to operation 412 where the interpolated adjustments are applied to the non-tested channels of the auditory prosthesis.

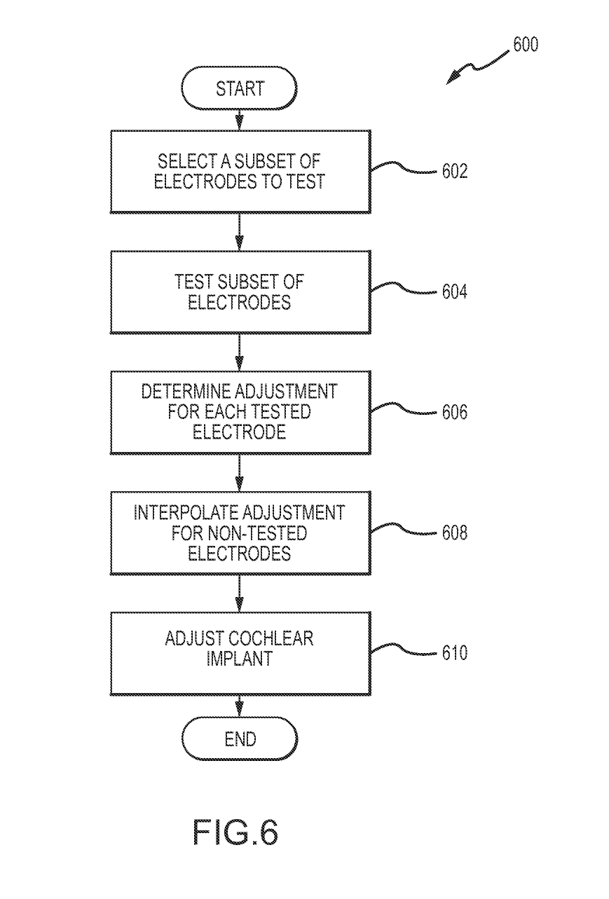

FIG. 6 is another exemplary method 600 for adjusting a cochlear implant. Flow begins at operation 602 where a subset of electrodes is selected for testing. In embodiments, the subset includes one or more sound channels. Each sound channel, in embodiments, can be related to one or more electrodes from the cochlear implant. For example, a cochlear implant includes twenty-two electrodes (labeled 1-22) that correspond to different sound channels. A subset of electrodes, such as the set {22, 20, 18, 16, 11, 8, 1}, is selected at operation 602. The subset can be selected according to various different methodologies.

Flow continues to operation 604 where tests are performed on the channels included in the selected subset. In one embodiment, testing is performed according to operations 402 and 404 of FIG. 4. In embodiments, a testing profile can be generated from testing results obtained during operation 604, such as, but not limited to, a PTA profile. In other embodiments, the testing results can be directly stored and later applied to a function or algorithm, for example, at operation 606 to determine adjustments for the tested electrodes, without the creation of a testing profile.

Upon determining the adjustments the tested electrodes, flow continues to operation 606 where adjustments are interpolated for the non-tested sound channels. In embodiments where the adjustments are for a cochlear implant, the adjustments can be made to one or more parameters of the electrical map for the electrode. For example, referring to the exemplary subset of electrodes {22, 20, 18, 16, 11, 8, 1}, an interpolated adjustment value for electrode 21 can be determined using the adjustment values for electrodes 22 and 20 as inputs to an interpolation function, adjustments for electrodes 12-15 can be interpolated using adjustment values for electrodes 16 and 11, etc.

After interpolating the adjustment values, flow continues to operation 610 where the cochlear implant is adjusted using the measured and interpolated values for one or more tested or non-tested electrodes.

The technologies described herein contemplate a more localized method, that is, adjustments that have the least effect on other sound frequencies and related electrodes. The embodiments recalculate the T-level as illustrated in by the graph 500. The new curve 504 is based on a new value T2 is such that it reaches level CL PTA at 30 dBHL. In the illustrated embodiment, the greatest change in electrical stimulation level is localized at soft level. In embodiments, a soft level is a level of sound that is soft, or quiet. For example, to a normal hearing person, 30-50 dBHL are soft, 50-70 dBHL are normal, and sounds in excess of 70 dbHL are loud. There is some impact across the full acoustic range with the current definition of the electrical mapping function; however, the impact is minimized in this exemplary embodiment.

A difficulty with the prior art approach is that changes in loudness perception can occur, particularly at the higher presentation levels. For example, to obtain an aided threshold at 30 dBHL, the mapping parameters for the particular electrode must be adjusted to stimulate at level CL_PTA for an acoustic input of 30 dB. By adding a 10 dB channel gain, this will shift the whole mapping curve to the left and distort loudness perception at higher presentation levels. This is a bigger problem for speech understanding for cochlear implant recipients and/or calls for yet further fine tuning of overall volume levels. In embodiments, rather than increasing loudness, it is beneficial to adjust other parameters, such as T-level, particularly with respect to aiding a recipient's ability to understand spoken language.

Exemplary cochlear implants have a multitude of channels (e.g., twenty or more). Pure tone testing is typically performed at fewer frequencies (typically six channels). Interpolation can be employed to “speed up” the fine tuning process. By using interpolation, rather than testing every sound channel, a subset of sound channels can be tested to determine fine tuning adjustments for those channels. The determined fine tuning adjustments can then be used to interpolate adjustments for the non-tested channels. To further improve the fine tuning, electrical adjustments to different sound channels may be interpolated based on the auditory testing. In embodiments, an electrical map can be used to determine how much electrical stimulation is associated with each auditory test. Based on the map, electrical adjustments can be determined based upon the auditory stimulation. Furthermore, interpolation can be used with the electrical map to determine electrical adjustments for channels not tested. As such, the efficiency of the fine tuning process is increased without sacrificing quality.

Performing a fine tuning on all of the sound channels of the device can be a long and taxing process and requires the recipient to visit a trained audiologist. In embodiments, the fine tune protocols disclosed herein could be performed by software executing on a computer, such as a laptop, desktop, or other computing device that belongs to the recipient. Additionally, the fine tuning can be administered by professionals other than an expert CI audiologist.

Embodiments disclosed herein provide systems and methods that can be employed to efficiently fit an audio prosthesis for fine tuning to a recipient using interpolation. In such embodiments, values for sound channels can be adjusted without requiring the testing of each individual sound channel. The embodiments disclosed herein allow for quicker adjustments, thereby resulting in increased efficiency for an audiologist and reduced discomfort to an auditory prosthesis recipient. Furthermore, embodiments disclosed herein provide an easy fitting method that allows an auditory prosthesis recipient to perform self-fittings without requiring a visit to an audiologist.

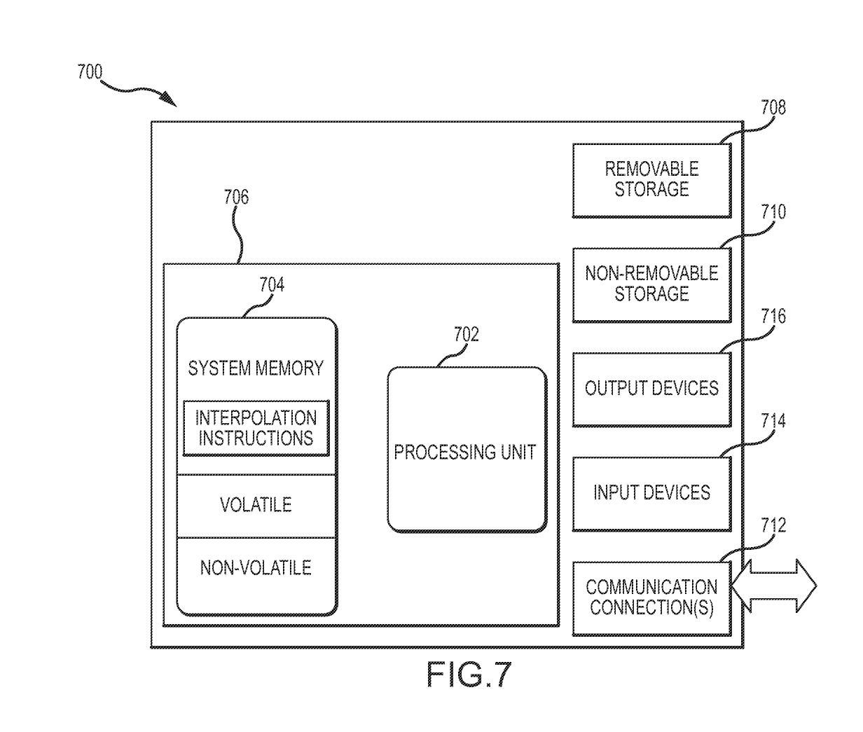

FIG. 7 illustrates one example of a suitable operating environment 700 in which one or more of the present embodiments can be implemented. This is only one example of a suitable operating environment and is not intended to suggest any limitation as to the scope of use or functionality. Other well-known computing systems, environments, and/or configurations that can be suitable for use include, but are not limited to, personal computers, server computers, hand-held or laptop devices, multiprocessor systems, microprocessor-based systems, programmable consumer electronics such as smart phones, network PCs, minicomputers, mainframe computers, tablets, distributed computing environments that include any of the above systems or devices, and the like.

In its most basic configuration, operating environment 700 typically includes at least one processing unit 702 and memory 704. Depending on the exact configuration and type of computing device, memory 704 (storing, among other things, instructions to implement and/or perform the modules and methods disclosed herein) can be volatile (such as RAM), non-volatile (such as ROM, flash memory, etc.), or some combination of the two. This most basic configuration is illustrated in FIG. 7 by dashed line 706. Further, environment 700 can also include storage devices (removable, 708, and/or non-removable, 710) including, but not limited to, magnetic or optical disks or tape. Similarly, environment 700 can also have input device(s) 714 such as touch screens, keyboard, mouse, pen, voice input, etc. and/or output device(s) 716 such as a display, speakers, printer, etc. Also included in the environment can be one or more communication connections, 712, such as LAN, WAN, point to point, Bluetooth, RF, etc.

Operating environment 700 typically includes at least some form of computer readable media. Computer readable media can be any available media that can be accessed by processing unit 702 or other devices comprising the operating environment. By way of example, and not limitation, computer readable media can comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, solid state storage, or any other tangible medium which can be used to store the desired information. Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The operating environment 700 can be a single computer operating in a networked environment using logical connections to one or more remote computers. The remote computer can be a personal computer, a server, a router, a network PC, a peer device or other common network node, and typically includes many or all of the elements described above as well as others not so mentioned. The logical connections can include any method supported by available communications media. Such networking environments are commonplace in offices, enterprise-wide computer networks, intranets and the Internet.

In some embodiments, the components described herein comprise such modules or instructions executable by computer system 700 that can be stored on computer storage medium and other tangible mediums and transmitted in communication media. Computer storage media includes volatile and non-volatile, removable and non-removable media implemented in any method or technology for storage of information such as computer readable instructions, data structures, program modules, or other data. Combinations of any of the above should also be included within the scope of readable media. In some embodiments, computer system 700 is part of a network that stores data in remote storage media for use by the computer system 700.

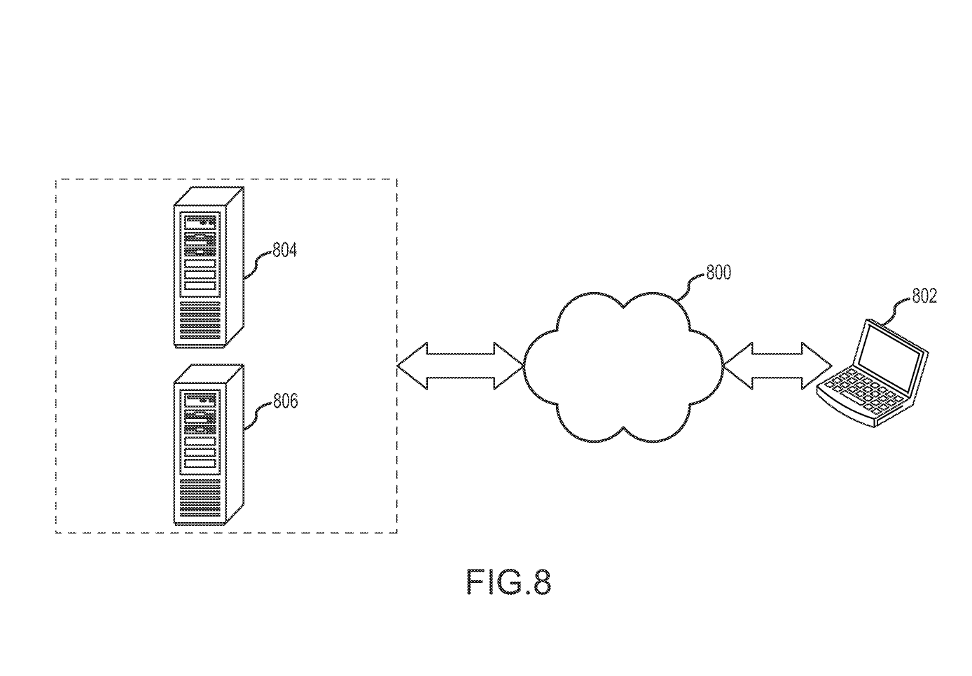

FIG. 8 is an embodiment of a network 800 in which the various systems and methods disclosed herein may operate. In embodiments, a client device, such as client device 802, may communicate with one or more servers, such as servers 804 and 806, via a network 808. In embodiments, a client device may be a laptop, a personal computer, a smart phone, a PDA, a netbook, or any other type of computing device, such as the computing device in FIG. 7. In embodiments, servers 1104 and 1106 may be any type of computing device, such as the computing device illustrated in FIG. 7. Network 1108 may be any type of network capable of facilitating communications between the client device and one or more servers 1104 and 1106. Examples of such networks include, but are not limited to, LANs, WANs, cellular networks, and/or the Internet.

In embodiments, the various systems and methods disclosed herein may be performed by one or more server devices. For example, in one embodiment, a single server, such as server 1104 may be employed to perform the systems and methods disclosed herein, such as the method for interpolating adjustment values. Client device 802 may interact with server 804 via network 808 in order to receive interpolated adjustment values during a fitting. In further embodiments, the client device 802 may also perform functionality disclosed herein, such as by applying testing signals and receive testing results of an auditory prosthesis, which can then be provided to servers 808 and/or 808.

In alternate embodiments, the methods and systems disclosed herein may be performed using a distributed computing network, or a cloud network. In such embodiments, the methods and systems disclosed herein may be performed by two or more servers, such as servers 804 and 806. Although a particular network embodiment is disclosed herein, one of skill in the art will appreciate that the systems and methods disclosed herein may be performed using other types of networks and/or network configurations.

The embodiments described herein can be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices can be employed to perform the functionality disclosed herein without departing from the scope of the disclosure.

This disclosure described some embodiments of the present technology with reference to the accompanying drawings, in which only some of the possible embodiments were shown. Other aspects can, however, be embodied in many different forms and should not be construed as limited to the embodiments set forth herein. Rather, these embodiments were provided so that this disclosure was thorough and complete and fully conveyed the scope of the possible embodiments to those skilled in the art.

Although specific embodiments were described herein, the scope of the technology is not limited to those specific embodiments. One skilled in the art will recognize other embodiments or improvements that are within the scope of the present technology. Therefore, the specific structure, acts, or media are disclosed only as illustrative embodiments. The scope of the technology is defined by the following claims and any equivalents therein.

Embodiments disclosed herein are described with respect to tuning a cochlear implant. Cochlear implants utilize multiple sounds channels, which, in embodiments, can be electrodes or groups of electrodes, and thus can benefit significantly from the technologies described. In other embodiments, the auditory prosthesis is an acoustic hearing aid, a bone conduction device, a middle ear auditory prosthesis, an auditory brainstem implant, or the like. While different types of auditory prostheses require different types of measurements and adjustments for tuning, one of skill in the art will appreciate that the embodiments disclosed herein can be employed to tune different types of auditory prostheses. However, for ease of illustration, and due to the benefits particular to utilization of the technology in cochlear implants, the various embodiments described herein will be described in the context of cochlear implants.

What is claimed is:

1. A method for fine tuning cochlear implant, the method comprising:
selecting a subset of electrodes for testing;
testing electrodes in the selected subset of electrodes to produce testing results,. wherein the testing comprises an auditory stimulation;
determining a first adjustment value based on the testing results, wherein the first adjustment value is an adjustment to an electrical output based upon an acoustic-to-electrical mapping;
based on the at least one adjustment value, determining an interpolated adjustment value for at least one non-tested electrode, wherein the interpolated adjustment value is an electrical output adjustment; and
adjusting a cochlear implant using the first adjustment value and the interpolated adjustment value, wherein the adjustment is performed after an initial fitting of the cochlear implant.

2. The method of claim 1, wherein the interpolated adjustment value is determined using linear interpolation.

3. The method of claim 1, wherein the interpolated adjustment value is based on only one tested electrode.

4. The method of claim 1, further comprising determining a second adjustment value based on the testing results.

5. The method of claim 4, wherein determining the interpolated adjustment value further comprises providing the first and the second adjustment values as input to an interpolation function to generate the interpolated adjustment value.

6. The method of claim 1, wherein the first adjustment value is applied to at least one tested electrode and the interpolated adjustment value is applied to a non-tested electrode.

7. The method of claim 1, wherein the electrical output adjustment comprises adjusting an electrical signal indicative of a T-level.

8. A method comprising:
outputting a first audio signal for a first sound channel;
receiving a first response indicative of the first audio signal being detected by a recipient, the first response indicating a first detection level;
determining that the first detection level does not meet a target threshold; and
adjusting a first map parameter set of the first sound channel such that the first detection level meets the target threshold.

9. The method of claim 8, further comprising:
outputting a second audio signal for a second sound channel;
receiving a second response indicative of the second audio signal being detected by the recipient, the second response indicating a second detection level;
determining that the second detection level does not meet the target threshold;
adjusting a second map parameter set of the second sound channel such that the second detection level meets the target threshold; and
adjusting a third map parameter set of a third sound channel based at least in part on the adjustment to the first map parameter set and the second map parameter set.

10. The method of claim 9, wherein the third sound channel corresponds to a third electrode disposed between a first electrode corresponding to the first sound channel and a second electrode corresponding to the second sound channel.

11. The method of claim 8, wherein the determination is based at least in part on a Pure-Tone Audiogram (PTA) profile.

12. The method of claim 9, wherein the target threshold relates to is a pure tone detection threshold expressed in dBHL.

13. The method of claim 8, wherein the first map parameter set is selected from a group consisting of:
microphone sensitivity;
channel gain;
T-SPL; and
channel T-level.

14. The method of claim 9, wherein adjusting the third sound channel further comprises:
determining a first adjustment value set for the first map parameter set;
determining a second adjustment value set for the second map parameter set;
inputting the first adjustment value set to an interpolation function to determine an interpolated adjustment value set; and
adjusting the third map parameter set using the interpolated adjustment value set.

15. A computer storage medium encoding computer executable instructions that, when executed by at least one processor, perform a method comprising:
outputting a first audio signal at a first sound channel of an auditory prosthesis;
receiving a first response indicative of the first signal being detected by a recipient;
determining that the first audio signal does not meet a detection threshold;
adjusting the first sound channel using a first adjustment value; and
adjusting a second sound channel based on a second adjustment value, wherein the second adjustment value is based at least in part on the first adjustment value.

16. The computer storage medium of claim 15, wherein the method further comprises:
outputting a second audio signal at a third sound channel;
receiving a second response indicative of the second audio signal being detected by the recipient;
determining that the second audio signal does not meet the detection threshold;
adjusting the third sound channel using a third adjustment value; and
adjusting the second sound channel using the second adjustment value, wherein the second adjustment value is based at least in part on the first adjustment value and the third adjustment value.

17. The computer storage medium of claim 15, adjusting the first sound processing channel comprises adjusting at least one of a current, a voltage, a frequency, a power, an intensity, and an amplitude.

18. The computer storage medium of claim 15, wherein the method further comprises determining the second adjustment value using interpolation.

19. The computer storage medium of claim 18, wherein determining the second adjustment value using interpolation further comprises inputting the first adjustment value to a linear interpolation function to generate the second adjustment value.

20. The computer storage medium of claim 15, wherein adjusting second sound channel comprises adjusting at least one parameter for an electrical map for the second sound channel.
